(19) United States
(12) Patent Application Publication
(10) Pub. No.: US 2008/0269353 A1
(43) Pub. Date: Oct. 30, 2008

(54) Preventative Composition For Ophthalmic Use

(75) Inventors: Koichi Takada, Osaka (JP); Akio Kimura, Osaka (JP); Mikiko Okemoto, Osaka (JP)

Correspondence Address: FRISHAUF, HOLTZ, GOODMAN & CHICK, PC, 220 Fifth Avenue, 16TH Floor, NEW YORK, NY, 10001-7708, US

(73) Assignee: SANTEN PHARMACEUTICAL CO., LTD., Osaka-shi, Osaka (JP)

(21) Appl. No.: 11/988,613

(22) PCT Filed: Jul. 13, 2006
(86) PCT No.: PCT/JP2006/313944

§ 371 Date: Jan. 11, 2008

(30) Foreign Application Priority Data
Jul. 13, 2005 (JP) 2005-204472

(51) Int. Cl. A61K 47/06; A61K 47/02

(52) U.S. Cl. 514/769

(57) ABSTRACT

An object of the present invention is to prevent the generation of chlorine dioxide in a liquid preparation for ophthalmic use containing a chlorite. A liquid preparation for ophthalmic use containing a preservative composition for ophthalmic use comprising a chlorite and at least one stabilizer selected from the following 1) to 7): 1) creatinine; 2) geraniol; 3) glucose; 4) tocopherol acetate; 5) oxyquinoline sulfate; 6) a sugar alcohol; and 7) a polyoxyethylene sorbitan fatty acid ester can prevent the generation of chlorine dioxide, and is therefore excellent in safety and exhibits a sustained preservative effect for a prolonged period of time.

### TECHNICAL FIELD

The present invention relates to a preservative composition for ophthalmic use comprising a chlorite and a stabilizer for the chlorite, and a liquid preparation for ophthalmic use comprising the composition.

2. Background Art

Eye drops are directly administered to a sensitive organ of the human body called eyes, and further, contact lenses are also used in a situation in which they are directly contacted with the eyes. Therefore, attention should be paid to the components to be formulated in eye drops or saline solutions for contact lenses from the viewpoint of safety. In particular, irritation to the eyes, side effects and the like should be taken into consideration.

As a preservative component of eye drops, for example, benzalkonium chloride, benzethonium chloride, sorbic acid and the like, and as a preservative component of saline solutions for contact lenses, for example, polyhexamethylene biguanide (PHMB), Polyquad, hydrogen peroxide, Purite (stabilized chlorine dioxide) and the like have been put to practical use.

Benzalkonium chloride or benzethonium chloride has an excellent preservative effect, however, when its concentration is increased, a corneal disorder may be sometimes caused. Therefore, its concentration when used is restricted to a certain degree. Further, these preservatives may sometimes cause alteration of the effects due to chemical reaction with an acidic additive, and also have a property to be easily adsorbed on eye drop containers or filtration filters. Sorbic acid is less likely to be adsorbed on eye drop containers, however, its preservative effect is not sufficient, and further, its stability is decreased depending on the pH, therefore, the formulation thereof in eye drops is restricted to a certain degree. On the other hand, peroxide preservatives such as hydrogen peroxide exhibit excellent disinfecting and washing effects when they are formulated in saline solutions for contact lenses, however, they are required to be neutralized because of their irritativeness.

A chlorite is a compound having ClO₂⁻ ion, and particularly sodium chlorite is used as a disinfectant for tap water or the like. However, it is known that when a chlorite is decomposed, chlorine dioxide having a strong oxidizing action is generated to cause irritation to the eyes, skin, respiratory tract, etc. On the other hand, JP-A-3-164402 discloses an invention relating to a method for producing chlorine dioxide and a disinfectant composition, in which chlorine dioxide is generated from a chlorine dioxide precursor such as a chlorite using a transition metal and thereby contact lenses and the like are disinfected and washed taking advantage of the strong disinfecting action of chlorine dioxide.

### DISCLOSURE OF THE INVENTION

### Problems to be Solved

Chlorine dioxide is a strong oxidizing agent like hydrogen peroxide and is known to have high irritativeness to the eyes, skin, etc. Therefore, when a chlorite is used as a preservative component for ophthalmic use, it is an important subject to prevent the generation of chlorine dioxide thereby to increase safety.

### Means for Solving the Problems

The present inventors studied stabilizers for preventing the generation of chlorine dioxide in various manners, and as a result, they found that a liquid preparation for ophthalmic use containing a preservative composition for ophthalmic use comprising a chlorite as a preservative component, and at least one stabilizer selected from creatinine, geraniol, glucose, tocopherol acetate, oxyquinoline sulfate, a sugar alcohol and a polyoxyethylene sorbitan fatty acid ester can significantly prevent the generation of chlorine dioxide, and is therefore excellent in safety and exhibits a sustained preservative effect for a prolonged period of time.

That is, the present invention is directed to:

(1) a preservative composition for ophthalmic use comprising a chlorite and at least one stabilizer selected from the following 1) to 7):

1) creatinine;

2) geraniol;

3) glucose;

4) tocopherol acetate;

5) oxyquinoline sulfate;

6) a sugar alcohol; and

7) a polyoxyethylene sorbitan fatty acid ester;

(2) the preservative composition for ophthalmic use according to the above (1), wherein the chlorite is sodium chlorite;

(3) the preservative composition for ophthalmic use according to the above (1), wherein the sugar alcohol is mannitol, sorbitol or xylitol;

(4) the preservative composition for ophthalmic use according to the above (1), wherein the polyoxyethylene sorbitan fatty acid ester is polysorbate 80;

(5) a liquid preparation for ophthalmic use comprising the preservative composition for ophthalmic use according to any one of the above (1) to (4); and

(6) a liquid preparation for ophthalmic use comprising 0.0001 to 1% (w/v) of a chlorite and at least one stabilizer selected from the following 1) to 7):

1) 0.0001 to 5% (w/v) of creatinine;

2) 0.00001 to 0.05% (w/v) of geraniol;

3) 0.0001 to 5% (w/v) of glucose;

4) 0.0001 to 1% (w/v) of tocopherol acetate;

5) 0.00001 to 1% (w/v) of oxyquinoline sulfate;

6) 0.001 to 10% (w/v) of a sugar alcohol; and

7) 0.0001 to 10% (w/v) of a polyoxyethylene sorbitan fatty acid ester.

In the present invention, the chlorite as a preservative component is not particularly limited as long as it is a salt of chlorous acid, and examples thereof include alkali metal salts of chlorous acid such as sodium chlorite and potassium chlorite, alkaline earth metal salts of chlorous acid such as calcium chlorite, magnesium chlorite and barium chlorite, copper chlorite, lead chlorite, ammonium chlorite and the like, and more preferred chlorite is sodium chlorite.

The concentration of the chlorite in the liquid preparation for ophthalmic use is preferably in the range of from 0.00001 to 1 s (w/v), more preferably from 0.0001% to 0.1% (w/v).

In the present invention, examples of the stabilizer capable of stabilizing the chlorite as a preservative component thereby to prevent the generation of chlorine dioxide include the following 7 substances.

1) creatinine

2) geraniol

3) glucose

4) tocopherol acetate

5) oxyquinoline sulfate

6) a sugar alcohol

7) a polyoxyethylene sorbitan fatty acid ester

Examples of the sugar alcohol include mannitol, sorbitol, xylitol, sucrose and the like, and more preferred is mannitol. Examples of the polyoxyethylene sorbitan fatty acid ester include polysorbate 80 [polyoxyethylene sorbitan monooleate], polysorbate 60 [polyoxyethylene sorbitan monostearate], polysorbate 40 [polyoxyethylene sorbitan monopalmitate], polyoxyethylene sorbitan monolaurate, polyoxyethylene sorbitan trioleate, polysorbate 65 [polyoxyethylene sorbitan tristearate] and the like, and more preferred one is polysorbate 80.

The concentration of the stabilizer in the liquid preparation for ophthalmic use is preferably in the range of from 0.0001 to 59 (w/v) if it is creatinine, from 0.00001 to 0.05% (w/v) if it is geraniol, from 0.0001 to 5% (w/v) if it is glucose, from 0.0001 to 1% (w/v) if it is tocopherol acetate (α, β, γ, δ), from 0.00001 to 1% (w/v) if it is oxyquinoline sulfate, from 0.001 to 10% (w/v) if it is a sugar alcohol, from 0.0001 to 10% (w/v) if it is a polyoxyethylene sorbitan fatty acid ester.

The above-mentioned 7 stabilizers may be used alone or in combination thereof.

The liquid preparation for ophthalmic use of the present invention is preferably used as, for example, an eye drop or a saline solution for contact lenses, and can be prepared by widely used methods.

Further, in the liquid preparation for ophthalmic use of the present invention, a drug, a tonicity agent, a buffer, a pH adjusting agent, a viscosity increasing agent or the like can be appropriately formulated as needed.

The drug to be formulated in the liquid preparation for ophthalmic use of the present invention is not particularly limited, and examples thereof include antiglaucoma agents (such as timolol, prostaglandin derivatives and carbonate dehydratase inhibitors), a variety of vitamins (such as vitamin B2, vitamin B6, vitamin B12, vitamin E and panthenol), decongestants (such as tetrahydrozoline hydrochloride and naphazoline hydrochloride), antiinflammatory drugs (such as diclofenac, indometacin, fluorometholone, pranoprofen, glycyrrhizinate dipotassium and ε-aminocaproic acid), antihistamines (such as chlorpheniramine maleate and diphenhydramine hydrochloride), antiallergic drugs (such as sodium cromoglicate), antimicrobial drugs (such as quinolone antimicrobial agents, cephalosporins, sulfacetamide sodium and sulfamethoxazole), amino acids (such as potassium L-aspartate, aminoethylsulfonic acid and sodium chondroitin sulfate), diagnostic reagents (such as fluorescein sodium), sodium hyaluronate, neostigmine methylsulfate and the like.

Examples of the tonicity agent include glycerin, propylene glycol, polyethylene glycol, sodium chloride, potassium chloride, calcium chloride, magnesium chloride and the like.

Examples of the buffer include phosphates such as sodium phosphate, sodium dihydrogen phosphate, disodium hydrogen phosphate, potassium phosphate, potassium dihydrogen phosphate and dipotassium hydrogen phosphate; borates such as sodium borate and potassium borate; citrates such as sodium citrate and disodium citrate; acetates such as sodium acetate and potassium acetate; carbonates such as sodium carbonate, sodium hydrogen carbonate; trometamol and epsilon-aminocaproic acid, and the like.

Examples of the pH adjusting agent include hydrochloric acid, citric acid, phosphoric acid, acetic acid, sodium hydroxide, potassium hydroxide, sodium carbonate, sodium hydrogen carbonate and the like.

Examples of the viscosity increasing agent include hydroxypropylmethyl cellulose, hydroxypropyl cellulose, polyvinyl alcohol, carboxyvinyl polymer, polyvinylpyrrolidone and the like.

The pH of the liquid preparation for ophthalmic use of the present invention is preferably in the range of from 3 to 9, particularly from 5 to 8.

### ADVANTAGE OF THE INVENTION

The liquid preparation for ophthalmic use containing the preservative composition for ophthalmic use comprising a chlorite and at least one stabilizer selected from creatinine, geraniol, glucose, tocopherol acetate, oxyquinoline sulfate, a sugar alcohol and a polyoxyethylene sorbitan fatty acid ester can prevent the generation of chlorine dioxide, and is therefore excellent in safety and exhibits a sustained preservative effect for a prolonged period of time.

### BEST MODE FOR CARRYING OUT THE INVENTION

### 1. Test for prevention of generation of chlorine dioxide

### (1) Sample Preparation

Test solution 1

7 mg of a chlorite, 500 mg of creatinine and 20 mg of sodium hydrogen phosphate were dissolved in about 80 mL of purified water, and the pH of the solution was adjusted to 7.0 with dilute hydrochloric acid or sodium hydroxide. Then, the total volume was made up to 100 mL with purified water, whereby Test solution 1 was obtained.

Test solutions 2 to 6

Test solutions 2 to 6 were obtained by carrying out the same procedure as that of Test solution 1 except that 5 mg of geraniol, 200 mg of glucose, 2 g of mannitol, 100 mg of polysorbate 80 and 10 mg of oxyquinoline sulfate were used, respectively, in place of 500 mg of creatinine in Test solution 1.

### Test Solution 7

140 mg of d-α-tocopherol acetate and 100 mg of polysorbate 80 were mixed, and 7 mg of a chlorite, 20 mg of sodium hydrogen phosphate and about 80 mL of purified water were added thereto and dissolved. Then, the pH of the solution was adjusted to 7.0 with dilute hydrochloric acid or sodium hydroxide, and the total volume was made up to 100 mL with purified water, whereby Test solution 7 was obtained.

### Comparative Test Solution 1

7 mg of a chlorite and 20 mg of sodium hydrogen phosphate were dissolved in about 80 mL of purified water, and the pH of the solution was adjusted to 7.0 with dilute hydrochloric acid or sodium hydroxide. Then, the total volume was made up to 100 mL with purified water, whereby Comparative test solution 1 was obtained.

### (2) Test Method and Results

1) Determination of Chlorine Dioxide by DPD Method (for 14 days)

By using Test solutions 1 to 5 and Comparative test solution 1, the concentration (ppm) of generated chlorine dioxide was determined by the DPD method described in the Standard Methods for Examination of water. These test results are shown in Table 1.

TABLE 1

| | | Concentration of chlorine | |
|---|---|---|---|
| | | dioxide (ppm) | |
| | Stabilizer | After 7 days | After 14 days |
| Test solution 1 | Creatinine | N.D. *¹ | N.D. |
| Test solution 2 | Geraniol | N.D. | N.D. |
| Test solution 3 | Glucose | N.D. | N.D. |
| Test solution 4 | Mannitol | N.D. | N.D. |
| Test solution 5 | Polysorbate 80 | N.D. | N.D. |
| Comparative test | | 0.087 | 0.15 |
| solution 1 | | | |

Note)
*¹ N.D. indicates that the value is 0.02 ppm or less (measuring range of DPD method: 0.02 to 5 ppm).

2) Confirmation of Existence of Chlorine Dioxide with Detector Tube (for 14 Days)

Determination by the DPD method could not be carried out for Test solutions 6 and 7 because they were colored, therefore, the existence of chlorine dioxide was confirmed using a simple test method with a detector tube. To be more specific, chlorine dioxide in Test solutions 6 and 7 and Comparative test solution 1 was vaporized under reduced pressure, and the existence of chlorine dioxide was confirmed with a detector tube. These test results are shown in Table 2.

TABLE 2

| | | Concentration of |
|---|---|---|
| | | chlorine dioxide |
| | Stabilizer | after 14 days (ppm) |
| Test solution 6 | Oxyquinoline | N.D. *² |
| | sulfate | |
| Test solution 7 | D-α-tocopherol | N.D. |
| | acetate and | |
| | Polysorbate 80 | |
| Comparative test | | 0.2 |
| solution 1 | | |

Note)
*² N.D. indicates that the existence of chlorine dioxide was not observed (measuring range of detector tube: 0.05 to 0.6 ppm).

### (3) Discussion

As apparent from Table 1 and Table 2, the liquid preparations for ophthalmic use containing the preservative composition for ophthalmic use comprising a chlorite and each stabilizer (creatinine, geraniol, glucose, mannitol, polysorbate 80, oxyquinoline sulfate and d-α-tocopherol acetate) can significantly prevent the generation of chlorine dioxide and are therefore excellent in safety and cause less irritation to the eyes.

### 2. Test for Preservative Effectiveness

### (1) Test for Preservative Effectiveness Using P. aeruginosa and C. albicans

### 1) Sample

The above-mentioned Test solutions 1 to 7 were used.

### 2) Test Method and Results

A test for preservative effectiveness was carried out according to Preservatives-Effectiveness Tests described in the Japanese Pharmacopoeia Fourteenth Edition. As test microorganisms, Pseudomonas aeruginosa (P. aeruginosa) and Candida albicans (C. albicans) were used, and the viable cell numbers after 7 days and 14 days were measured. The survival rate (%) of the microorganisms was calculated according to the following calculation equation.

Survival rate (%)=[(Viable cell number when sampling)/(Initial cell number)]×100

These test results are shown in Table 3.

TABLE 3

| | | | Survival rate (%) | |
|---|---|---|---|---|
| | | Test | After | After |
| | Stabilizer | microorganism | 7 days | 14 days |
| Test solution 1 | Creatinine | P. aeruginosa | N.D. *³ | N.D. |
| | | C. albicans | 20.7 | 10.0 |
| Test solution 2 | Geraniol | P. aeruginosa | N.D. | N.D. |
| | | C. albicans | 3.3 | 0.5 |
| Test solution 3 | Glucose | P. aeruginosa | N.D. | N.D. |
| | | C. albicans | N.D. | N.D. |
| Test solution 4 | Mannitol | P. aeruginosa | N.D. | N.D. |
| | | C. albicans | 65.3 | 54.0 |
| Test solution 5 | Polysorbate 80 | P. aeruginosa | N.D. | N.D. |
| | | C. albicans | 66.7 | 66.7 |
| Test solution 6 | Oxyquinoline | P. aeruginosa | N.D. | N.D. |
| | sulfate | C. albicans | 5.1 | 0.1 |
| Test solution 7 | D-α-tocopherol | P. aeruginosa | N.D. | N.D. |
| | acetate and | C. albicans | 58.7 | 17.3 |
| | Polysorbate 80 | | | |

Note)
*³ N.D. indicates that microorganisms were not detected.

(2) Test for preservative effectiveness using A. niger

### 1) Sample Preparation

### Test Solution 8

20 mg of a chlorite, 5 mg of geraniol and 200 mg of sodium hydrogen phosphate were dissolved in purified water, and the pH of the solution was adjusted to 6.5 with dilute hydrochloric acid or sodium hydroxide. Then, the total volume was made up to 100 mL with purified water, whereby Test solution 8 was obtained.

Test solutions 9 and 10

Test solutions 9 and 10 were obtained by carrying out the same procedure as that of Test solution 8 except that 50 mg of creatinine and 2 g of mannitol were used, respectively, in place of 5 mg of geraniol in Test solution 8.

Test solutions 11 and 12

Test solutions 11 and 12 were obtained by carrying out the same procedure as that of Test solution 8 except that the amount of a chlorite was changed from 20 mg in Test solution 8 to 50 mg and 7 mg, respectively.

### 2) Test Method and Results

By using Test solutions 8 to 12, a test for preservative effectiveness was carried out. The test for preservative effectiveness was carried out according to Preservatives-Effectiveness Tests described in the Japanese Pharmacopoeia Fourteenth Edition. As a test microorganism, Aspergillus niger (A. niger) was used, and the viable cell numbers after 7 days, 14 days and 28 days were measured. The acceptance criteria of the Japanese Pharmacopoeia (JP) and the US Pharmacopoeia (USP) for Category IA into which eye drops are classified are shown below.

Acceptance criteria of JP (fungi): The viable cell numbers after 14 days and 28 days are the same as or lower than the inoculated cell number.

Acceptance criteria of USP (fungi): The viable cell numbers after 7 days, 14 days and 28 days do not increase from the inoculated cell number.

These test results are shown in Table 4.

TABLE 4

| | | | Test | Acceptance | Acceptance |
|---|---|---|---|---|---|
| | Stabilizer | Chlorite (mg) | microorganism | result (JP) | result (USP) |
| Test solution 8 | Geraniol | 20 | A. niger | Accepted | Accepted |
| Test solution 9 | Creatinine | 20 | A. niger | Accepted | Accepted |
| Test solution 10 | Mannitol | 20 | A. niger | Accepted | Accepted |
| Test solution 11 | Geraniol | 50 | A. niger | Accepted | Accepted |
| Test solution 12 | Geraniol | 7 | A. niger | Accepted | Accepted |

(3) Test for Preservative Effectiveness Using 5 species of Microorganisms

### 1) Sample Preparation

### Test Solution 13

7 mg of a chlorite, 5 mg of geraniol, 400 mg of potassium chloride, 100 mg of sodium chloride and 1 g of boric acid were dissolved in purified water, and the pH of the solution was adjusted to 7.5 with dilute hydrochloric acid or sodium hydroxide. Then, the total volume was made up to 100 mL with purified water, whereby Test solution 13 was obtained.

### Test Solution 14

Test solution 14 was obtained by carrying out the same procedure as that of Test solution 13 except that 2 g of mannitol was used in place of 5 mg of geraniol in Test solution 13. Comparative test solution 2

400 mg of potassium chloride, 100 mg of sodium chloride and 1 g of boric acid were dissolved in purified water, and the pH of the solution was adjusted to 7.5 with dilute hydrochloric acid or sodium hydroxide. Then, the total volume was made up to 100 mL with purified water, whereby Comparative test solution 2 was obtained.

### 2) Test Method and Results

By using Test solutions 13 to 14 and Comparative test solution 2, a test for preservative effectiveness was carried out. The test for preservative effectiveness was carried out according to Preservatives-Effectiveness Tests described in the Japanese Pharmacopoeia Fourteenth Edition. As test microorganisms, Escherichia coli (E. coli), Pseudomonas aeruginosa (P. aeruginosa), Staphylococcus aureus (S. aureus), Candida albicans (C. albicans) and Aspergillus niger (A. niger) were used, and the viable cell numbers after 14 days and 28 days were measured. The survival rate (%) of the microorganisms was calculated according to the following calculation equation.

Survival rate (%)=[(Viable cell number when sampling)/(Initial cell number)]×100

These test results are shown in Table 5.

TABLE 5

| | | | Survival rate (%) | |
|---|---|---|---|---|
| | | Test | After | After |
| | Stabilizer | microorganism | 14 days | 28 days |
| Test solution 13 | Geraniol | E. coli | 0.0 | 0.0 |
| | | P. aeruginosa | 0.0 | 0.0 |
| | | S. aureus | 0.0 | 0.0 |
| | | C. albicans | 0.9 | 0.0 |
| | | A. niger | 0.6 | 0.5 |
| Test solution 14 | Mannitol | E. coli | 0.0 | 0.0 |
| | | P. aeruginosa | 0.0 | 0.0 |
| | | S. aureus | 0.0 | 0.0 |
| | | C. albicans | 0.0 | 0.0 |
| | | A. niger | 0.1 | 0.1 |
| Comparative test | | E. coli | 123.1 | 92.3 |
| solution 2 | | P. aeruginosa | 0.4 | 0.1 |
| | | S. aureus | 0.8 | 0.0 |
| | | C. albicans | 47.4 | 0.0 |
| | | A. niger | 0.7 | 2.6 |

### (4) Discussion

As apparent from Tables 3 to 5, the liquid preparations for ophthalmic use containing the preservative composition for ophthalmic use comprising a chlorite and each of the above-mentioned stabilizers exhibit an excellent preservative effect on various microorganisms such as Pseudomonas aeruginosa (gram-negative bacterium), Caidida (fungus) and Aspergillus niger (fungus).

### 3. Preparation Examples

Hereinafter, typical preparation examples will be shown.

Formulation example 1 (pH 7)
In 100 ml

| | | |
|---|---|---|
| Sodium hyaluronate | 100 | mg |
| Sodium chlorite | 7 | mg |
| Creatinine | 50 | mg |
| Sodium chloride | 850 | mg |
| Sodium dihydrogen phosphate | 200 | mg |

| | |
|---|---|
| Sodium hydroxide | q.s. |
| Dilute hydrochloric acid | q.s. |
| Sterile purified water | q.s. |

Formulation example 2 (pH 7)
In 100 ml

| | | |
|---|---|---|
| Sodium hyaluronate | 100 | mg |
| Sodium chlorite | 7 | mg |
| Geraniol | 5 | mg |
| Sodium chloride | 900 | mg |
| Sodium dihydrogen phosphate | 200 | mg |

| | |
|---|---|
| Sodium hydroxide | q.s. |
| Dilute hydrochloric acid | q.s. |
| Sterile purified water | q.s. |

Formulation example 3 (pH 7)
In 100 ml

| | | |
|---|---|---|
| Sodium hyaluronate | 100 | mg |
| Sodium chlorite | 7 | mg |
| Mannitol | 2 | g |
| Sodium dihydrogen phosphate | 200 | mg |

| | |
|---|---|
| Sodium hydroxide | q.s. |
| Dilute hydrochloric acid | q.s. |
| Sterile purified water | q.s. |

Formulation example 4 (pH 7)
In 100 ml

| | | |
|---|---|---|
| Potassium chloride | 100 | mg |
| Sodium chloride | 400 | mg |
| Sodium chlorite | 7 | mg |
| Geraniol | 5 | mg |
| Boric acid | 1 | g |

| | |
|---|---|
| Sodium hydroxide | q.s. |
| Dilute hydrochloric acid | q.s. |
| Sterile purified water | q.s. |

1. A preservative composition for ophthalmic use comprising a chlorite and at least one stabilizer selected from the following 1) to 7):
1) creatinine;
2) geraniol;
3) glucose;
4) tocopherol acetate;
5) oxyquinoline sulfate;
6) a sugar alcohol; and
7) a polyoxyethylene sorbitan fatty acid ester.

2. The preservative composition for ophthalmic use according to claim 1, wherein the chlorite is sodium chlorite.

3. The preservative composition for ophthalmic use according to claim 1, wherein said stabilizer is mannitol, sorbitol or xylitol.

4. The preservative composition for ophthalmic use according to claim 1, wherein said stabilizer is polysorbate 80.

5. A liquid preparation for ophthalmic use comprising the preservative composition for ophthalmic use according to claim 1.

6. A liquid preparation for ophthalmic use comprising 0.00001 to 1% (w/v) of a chlorite and at least one stabilizer selected from the following 1) to 7):
1) 0.0001 to 5% (w/v) of creatinine;
2) 0.00001 to 0.05% (w/v) of geraniol;
3) 0.0001 to 5% (w/v) of glucose;
4) 0.0001 to 1% (w/v) of tocopherol acetate;
5) 0.00001 to 1% (w/v) of oxyquinoline sulfate;
6) 0.001 to 10% (w/v) of a sugar alcohol; and
7) 0.0001 to 10% (w/v) of a polyoxyethylene sorbitan fatty acid ester.

7. A liquid preparation for ophthalmic use comprising the preservative composition for ophthalmic use according to claim 2.

8. A liquid preparation for ophthalmic use comprising the preservative composition for ophthalmic use according to claim 3.

9. A liquid preparation for ophthalmic use comprising the preservative composition for ophthalmic use according to claim 4.

10. A liquid preparation for ophthalmic use comprising the preservative composition for ophthalmic use according to claim 2, wherein said stabilizer is selected from the group consisting of mannitol, sorbitol, xylitol and polysorbate 80.

11. A method of stabilizing an ophthalmic preparation comprising admixing a chlorite and at least one stabilizer selected from the following 1) to 7) in said preparation:
1) creatinine;
2) geraniol;
3) glucose;
4) tocopherol acetate;
5) oxyquinoline sulfate;
6) a sugar alcohol; and
7) a polyoxyethylene sorbitan fatty acid ester.

12. The method according to claim 11, wherein the chlorite is sodium chlorite.

13. The method according to claim 11, wherein said stabilizer is mannitol, sorbitol or xylitol.

14. The method according to claim 11, wherein said stabilizer is polysorbate 80.

15. The method according to claim 11, wherein said preparation comprising 0.00001 to 1% (w/v) of a chlorite and at least one stabilizer selected from the following 1) to 7):
1) 0.0001 to 5% (w/v) of creatinine;
2) 0.00001 to 0.05% (w/v) of geraniol;
3) 0.0001 to 5% (w/v) of glucose;
4) 0.0001 to 1% (w/v) of tocopherol acetate;
5) 0.00001 to 1% (w/v) of oxyquinoline sulfate;
6) 0.001 to 10% (w/v) of a sugar alcohol; and
7) 0.0001 to 10% (w/v) of a polyoxyethylene sorbitan fatty acid ester.

16. The method according to claim 15, wherein the chlorite is sodium chlorite.

17. The method according to claim 15, wherein said stabilizer is mannitol, sorbitol or xylitol.

18. The method according to claim 15, wherein said stabilizer is polysorbate 80.

19. The method according to claim 16, wherein said stabilizer is mannitol, sorbitol or xylitol.

20. The method according to claim 16, wherein said stabilizer is polysorbate 80.
